(19) United States
(12) Patent Application Publication
(10) Pub. No.: US 2020/0033315 A1
(43) Pub. Date: Jan. 30, 2020

(54) METHODS FOR DETERMINING RESIDUAL SURFACTANT CONCENTRATIONS IN OIL AND WATER PHASES

(71) Applicant: ExxonMobil Research and Engineering Company, Annandale, NJ (US)

(72) Inventors: Fang Cao, Clinton, NJ (US); Shuji Luo, Basking Ridge, NJ (US); Marcia E. Dierolf, Oley, PA (US); Yunlong Zhang, Annandale, NJ (US)

(21) Appl. No.: 16/520,641

(22) Filed: Jul. 24, 2019

Related U.S. Application Data
(60) Provisional application No. 62/703,963, filed on Jul. 27, 2018.

(51) Int. Cl. G01N 33/28; G01N 33/18; G01N 31/12

(52) U.S. Cl. CPC G01N 33/287; G01N 31/12; G01N 33/2876; G01N 33/1833

(57) ABSTRACT

Methods for determining nitrogen and/or sulfur containing surfactant residual concentrations in oil and/or water phases of an oil and water system, particularly produced oil and water from an oil or gas well, are provided. The methods utilize pyro-chemiluminescence/pyro-fluorescence techniques which quantify the total residual surfactants in both oil and water phases. The methods may be applied broadly in areas where quantifying or monitoring surfactant concentrations in solution is required.

### CROSS-REFERENCE TO RELATED APPLICATIONS

This application claims priority to U.S. Provisional Application Ser. No. 62/703,963 filed Jul. 27, 2018, which is herein incorporated by reference in its entirety.

### FIELD

This disclosure relates to methods for determining nitrogen and/or sulfur containing surfactant residual concentrations in oil and water phases of an oil and water system, particularly a produced oil and water system from an oil or gas well. The methods utilize pyro-chemiluminescence/pyro-fluorescence and are fast, reliable and cost-effective.

### BACKGROUND

Surfactant is a chemical commonly used in the oil and gas industry and its applications include corrosion inhibition in production and transportation pipelines, enhancement of oil and gas production as a component in fracturing fluid additives, and enhanced oil recovery (EOR) as a component in the injected chemical solutions, and so forth. Measuring residual surfactant in produced water can be very useful for understanding the performance of surfactants in the field. However, because produced water contains various levels of total dissolved solid (TDS), other injected oil field chemicals (OFCs) (e.g., scale inhibitors, demulsifiers, biocides, friction reducers), and water-soluble organic acids and hydrocarbons, it is quite challenging to measure surfactant residuals. Conventional colorimetric methods (e.g., methyl orange, Hach) based on the complexation between cationic surfactants and the reagent can result in inaccuracies due to the different response of surfactants to the complexation reagent. Other techniques, such as liquid chromatography/mass spectrometry, and ultraviolet spectroscopy have their limitations as well, such as complex data interpretation, time consuming, expensive, and possible interactions of components from produced water.

Surfactants commonly used in the oil and gas industry contain nitrogen or sulfur, for example quaternary ammonium compounds, amides or sulfonates.

In view of the foregoing it would be desirable to provide a method for determining nitrogen and/or sulfur containing surfactant residual concentrations in oil and water phases of an oil and water system, particularly a produced oil and water system from an oil or gas well, that address one or more of the above deficiencies.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

### SUMMARY

The present disclosure provides methods for determining nitrogen and/or sulfur containing surfactant residual concentrations in oil and/or water phases of an oil and water system, particularly produced oil and water from an oil or gas well. The methods comprise pyro-chemiluminescence/pyro-fluorescence techniques which quantify the total residual surfactants in both oil and water phases. The methods may be applied broadly in areas where quantifying or monitoring surfactant concentrations in solution is required. The presently disclosed methods to provide a fast, reliable and cost-effective method to measure nitrogen and/or sulfur containing surfactant concentrations in both oil and water phases with relatively low detection limits.

In one aspect the present disclosure provides a method for determining the concentration of nitrogen and/or sulfur containing surfactants in oil and/or water phases of an oil and water system, comprising the steps of:
(a) partitioning the nitrogen and/or sulfur containing surfactant between the oil and water phases; and
(b) measuring the nitrogen and/or sulfur concentration in the oil and/or water phase by pyro-chemiluminescence and/or pyro-fluorescence.

The nitrogen may be measured by pyro-chemiluminescence. The sulfur may be measured by pyro-fluorescence.

The method may further comprise the step of separating at least a portion of the oil and/or water phases after partitioning the nitrogen and/or sulfur containing surfactant.

The method may further comprise the step of extracting at least a portion of the water phase with an organic solvent so as to transfer some or all of the nitrogen and/or sulfur containing surfactant to the organic solvent and measuring the concentration of nitrogen and/or sulfur in the organic solvent.

In some embodiments the oil and water system is a produced oil and water system from an oil or gas well. See, for example, ‘Produced Water: Environmental Risks and Advances in Mitigation Technologies, Lee, Kenneth and Neff, Jerry (Eds), Springer Science & Business Media, New York, N.Y., 2011, in particular Chapter 1, Section 2: Chemical Composition of Produced Water, which describes the many chemical constituents of produced water.

In some embodiments the water phase comprises one or more water soluble salts. The salts may be selected from alkali metal or alkaline earth salts such as alkali metal or alkaline earth halides, sulfates or bicarbonates. In a preferred embodiment the salt is sodium chloride. Other exemplary salts include potassium chloride, sodium bromide, calcium chloride, magnesium chloride, sodium sulfate and sodium bicarbonate.

In some embodiments the concentration of the one or more salts in the water phase is between about 0.1% and about 36% by weight. Preferably between about 0.5% and about 30% by weight.

Alternatively, the concentration of the one or more salts in the water phase is between about a few parts per thousand to about 300 parts per thousand.

In some embodiments the surfactant is selected from the group consisting of non-ionic surfactant, ionic surfactant, amphoteric surfactant, and combinations thereof.

In some preferred embodiments the nitrogen containing surfactant is a quaternary ammonium salt.

In other preferred embodiments the nitrogen containing surfactant is an amide.

In other preferred embodiments the sulfur containing surfactant is a sulfonate.

The concentration of surfactant in the oil and water phases may, independently, be between about 0.1 ppm and about 1000 ppm, preferably between about 0.5 ppm and about 500 ppm, more preferably between about 1.0 ppm and about 250 ppm.

The volume ratio of oil to water in the oil and water system may be between 0.1:1.0 and 1.0:0.1, preferably between 0.3:1.0 and 1.0:0.3.

The volume ratio of organic solvent to water may be between 0.1:1.0 and 1.0:0.1, preferably between 0.3:1.0 and 1.0:0.3

The organic solvent may be selected from aliphatic and aromatic hydrocarbon solvents, preferably aromatic hydrocarbon solvents such as xylene.

Examples of application of the presently disclosed method include, but are not limited to, measurement of corrosion inhibitor residuals in the field as part of a comprehensive corrosion monitoring program and monitoring surfactant residuals in produced waters to optimize surfactant applications during hydraulic fracturing operation.

Further features and advantages of the present disclosure will be understood by reference to the following drawings and detailed description.

### BRIEF DESCRIPTION OF THE DRAWINGS

FIG. 1 is a chart showing a calibration curve of measured nitrogen content in ppm plotted against surfactant concentration in o-xylene in ppm.

FIG. 2 is a chart showing a calibration curve of measured nitrogen content in ppm plotted against surfactant concentration in 1% brine in ppm.

FIG. 3 is a chart showing a calibration curve of measured nitrogen content in ppm plotted against surfactant concentration in 24% brine in ppm.

FIG. 4 is a chart showing a calibration curve of measured nitrogen content in ppm plotted against surfactant concentration in toluene in ppm.

FIG. 5 is a chart showing a calibration curve of measured nitrogen content in ppm plotted against surfactant concentration in 1% brine in ppm.

FIG. 6 is a chart showing a calibration curve of measured nitrogen content in ppm plotted against surfactant concentration in 24% brine in ppm.

FIG. 7 is a chart showing a calibration curve of measured nitrogen content in ppm plotted against surfactant concentration in MPN brine in ppm.

### DETAILED DESCRIPTION OF THE EMBODIMENTS

Before the present methods are disclosed and described, it is to be understood that unless otherwise indicated this disclosure is not limited to specific methods, compositions, components, or the like, as such may vary, unless otherwise specified. It is also to be understood that the terminology used herein is for the purpose of describing particular embodiments only and is not intended to be limiting.

It must also be noted that, as used in the specification and the appended claims, the singular forms ‘a’, ‘an’ and ‘the’ include plural referents unless otherwise specified. Thus, for example, reference to ‘surfactant’ may include more than one surfactant, and the like.

Throughout this specification, use of the terms “comprises” or “comprising” or grammatical variations thereon shall be taken to specify the presence of stated features, integers, steps or components but does not preclude the presence or addition of one or more other features, integers, steps, components or groups thereof not specifically mentioned.

All numerical values as used herein are modified by “about” or “approximately” the indicated value, and take into account experimental error and variations that would be expected by a person having ordinary skill in the art.

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

### Surfactant

The surfactant may be a nonionic surfactant, an ionic surfactant, an amphoteric surfactant, or any combination of surfactants.

As used herein, a “nonionic surfactant” refers to a surfactant in which the molecules forming the surfactant are uncharged. Examples of nonionic surfactants may include, but are not limited to, polyoxyethylene fatty acid amides, polyoxyethylene alkyl amines, alkylpyrrolidone, glucamides, alkylpolyglucosides, mono- and dialkanol amides, polyoxyethylene alcohol mono- or diamides and alkylamine oxides.

As used herein, an “ionic surfactant” refers to a surfactant in which the molecules forming the surfactant are charged. Suitable ionic surfactants may include, but are not limited to, sulfonates, sulfates, ammonium and sulfonium alkylated quaternary or ternary compounds, singly or attached to polymeric compounds.

Suitable anionic surfactants include, but are not limited to, those containing sulfonate and sulfate ions. Examples of anionic surfactants, include but are not limited to sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxy))-sulfosuccinate and alkyl sulfates such as sodium lauryl sulfate.

Suitable, cationic surfactants include, but are not limited to quaternary ammonium compounds such as benzalkonium chloride, benzethonium chloride, cetrimonium bromide, stearyl dimethylbenzyl ammonium chloride, and coconut amine. Examples of the anionic surfactants include but are not limited to alkane sulfonate salts, alpha-olefin sulfonate salts, sulfonate salts of higher fatty acid esters, higher alcohol sulfate ester salts, fatty alcohol ether sulfates salts, condensates of higher fatty acids and amino acids, and collagen hydrolysate derivatives. Examples of the cationic surfactants include but are not limited to alkyltrimethylammonium salts, dialkyldimethylammonium salts, alkyldimethylbenzylammonium salts, alkylpyridinium salts, alkylisoquinolinium salts, benzethonium chloride and acylamino acid type cationic surfactants.

As used herein, an “amphoteric surfactant” refers to a surfactant compound uniquely structured to function as cationic surfactants at acid pH and anionic surfactants at alkaline pH. Suitable amphoteric surfactants may include, but are not limited to, amino acid, betaine, sultaine, imidazoline type amphoteric surfactants, soybean phospholipid and yolk lecithin. Examples for amphoteric surfactants include, but are not limited to, sodium N-dodecyl-beta-alanine, sodium N-lauryl-beta-iminodipropionate, myristoamphoacetate and lauryl betaine.

Surfactants used in the oil and gas industry often contain nitrogen (e.g., amine based corrosion inhibitors) or sulfur (e.g., surfactants for fracturing fluid), while many other components in the produced water either do not contain nitrogen and sulfur, which can be excluded through baseline nitrogen and sulfur measurement/calibration, or can be selectively removed by special sample treatment. Since pyro-chemiluminescence and pyro-fluorescence have the capability to measure trace amounts of nitrogen and sulfur in organic solvent respectively, they may be used to determine the amount of total surfactants in solution.

### Pyro-Chemiluminescence and Pyro-Fluorescence

Pyro-chemiluminescence and pyro-fluorescence are fast and easy-to-use methods of respectively analyzing nitrogen and sulfur in a sample.

The sample is introduced into a reaction furnace where it is mixed with oxygen and burned, converting any organic nitrogen to nitric oxide and any organic sulfur to sulfur dioxide.

R—N+R—S+O₂->CO₂+H₂O+NO+SO₂+NOₓ

The products of combustion are routed through a membrane drier to remove all water and then to the detectors.

After passing through the membrane drier, the combustion gases go to a sulfur detector where they are exposed to UV light of a specific wavelength. The SO₂ absorbs this radiation, placing it into an excited state. The excited SO₂ relaxes, and emits excess energy (as fluorescent light) as a specific wavelength. This fluorescent emission is detected by a photomultiplier tube, completely specific for sulfur and is proportional to the amount of sulfur in the original sample.

SO₂+hv′->SO₂+hv″

The gas then travels towards the nitrogen detector, where NO and NOₓ are reacted with ozone (O₃) to form meta-stable NO₂*. When the excited nitrogen dioxide returns to normal state, it emits light at a specific wavelength which is then detected by the photomultiplier tube and is proportional to the amount of nitrogen in the original sample.

NO+NOₓ+O₃->NO₂*+O₂->NO₂+hv

### EXAMPLES

Details of the measurement procedure and sample handling and treatment process are described in the following. Pyro-chemiluminescence and pyro-luminesence measurements were performed using a PAC-ANTEK MultiTek instrument. Nitrogen was determined using ASTM method D4629-12 and sulfur determined using ASTM D5453.

### General Procedure of Surfactant Partitioning

Brine was prepared by dissolving sodium chloride in deionized water having a resistivity of 18 MΩ cm or greater. Aqueous surfactant solution was prepared using the brine and placed into a round-bottomed flask equipped with CO₂ flow. The solution was then saturated with CO₂ gas, while stirring, for at least 8 hours, so as to simulate the field condition of produced water and the pH then adjusted to the targeted value using HCl and NaHCO₃. The oil was then added and the mixture stirred vigorously for 60 minutes. The mixture was then transferred to an attached separation funnel to maintain the CO₂ blanket, and then left to separate for 90 minutes.

### General Procedure of Sample Preparation and Measurement

After partitioning, the oil phase was measured directly by a pyro-chemiluminescence and/or pyro-luminescence method to determine the nitrogen and/or sulfur content. If the boiling point of the surfactants of interest was much higher than the boiling point of dichloromethane (DCM), then the brine phase was extracted with DCM and then solvent transferred into o-xylene. The resulting o-xylene solution was then analyzed by pyro-chemiluminescence and/or pyro-luminescence to determine the nitrogen and/or sulfur content. If the boiling point of the surfactants of interest was close to that of DCM, then the brine phase was extracted with o-xylene and the resulting o-xylene solution was analyzed for nitrogen or sulfur content. To convert the measured nitrogen and/or sulfur content to surfactant concentration calibrations were established using a series of surfactant solutions of known concentrations.

### Example 1: Benzyldimethyldodecylammonium Chloride (Abbreviated as C12 Quat) Partitioning in Toluene and pH 5 Brine

### Surfactant Partitioning

1 wt. % NaCl brine was prepared by dissolving 0.3 g NaCl in 29.7 g deionized water. 0.003 g benzyldimethyldodecylammonium chloride (C12 quat) surfactant was then added to the brine to yield a 100 ppm C12 quat in 1 wt. % NaCl solution. The 30 ml volume solution was placed in a round-bottomed 3-neck flask equipped with CO₂ flow. The solution was then saturated with CO₂ gas for at least 8 hours, then the solution pH was adjusted to pH=5 using NaHCO₃ and HCl. An equal volume (30 ml) of toluene was added to the flask and the toluene/brine mixture stirred vigorously with a magnetic stir bar for 60 minutes. The mixture was then transferred to an attached separation funnel to maintain the CO2 blanket and left to separate for 90 minutes.

### Sample Preparation

After partition, 1 ml of the toluene phase was extracted from the top of the separation funnel using a pipette and submitted for quantitative total nitrogen measurement by pyro-chemiluminescence. 10 g of the brine phase was collected from the bottom of the separation funnel, mixed with equal weight (10 g) of dichloromethane in a separate separation funnel and the mixture vigorously shaken by hand for two minutes and then left to separate for 10 minutes. 5 g of the lower dichloromethane layer was collected in a small glass vial and the dichloromethane solvent removed by gently blowing a stream of nitrogen into the vial. The residue on the bottom of the vial was then re-dissolved in 5 g of o-xylene. The resulting o-xylene solution was analyzed for total nitrogen by pyro-chemiluminescence.

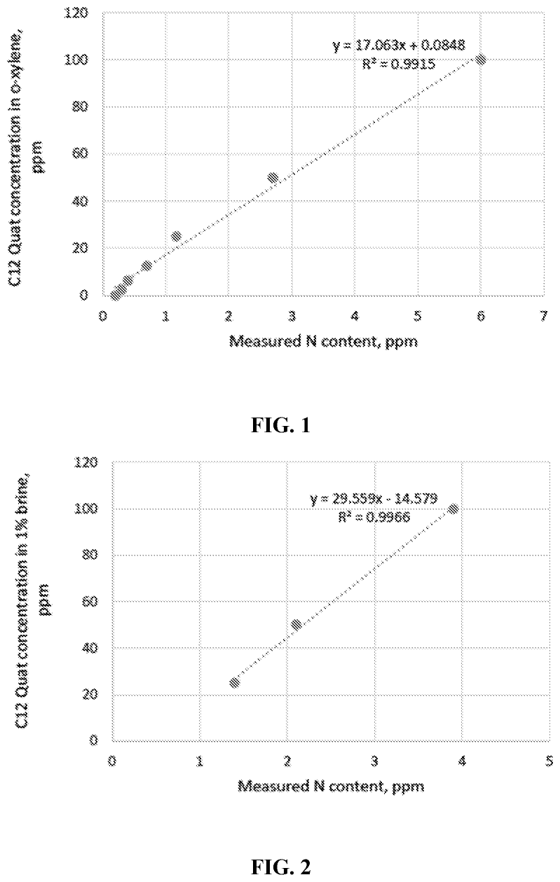

The partitioning experiments were conducted using the above procedure for 100 ppm C12 quat in pH=5 brine with equal volume of toluene at ambient temperature. The calibration curves for C12 quat in o-xylene, 1 wt. % NaCl and 24 wt. % NaCl are shown in FIGS. 1-3, respectively.

The measured nitrogen contents for the toluene and brine samples after partitioning are listed in Table 1. Corresponding C12 quat concentrations are calculated based on this data and the corresponding calibrations in FIG. 1-3. The partition of C12 quat in the brine phase decreased dramatically with increasing salinity in the brine phase. This is consistent with the significant increase of C12 quat concentration in the toluene phase with increasing salinity in the brine. This observation is consistent with the fat that increasing salinity will decrease the partitioning of ionic inhibitors into the water phase and can be rationalized by the screening effect of the salt. Dissociated salt ions screen the charges on the head group of C12 quat, hence drive the surfactant into the oil phase. One additional observation is that the measured C12 quat residual concentration in the oil and brine phases did not add up to the original starting concentration of 100 ppm in both partition tests with different brine salinities. It is believed that the majority of the missing surfactants remain at the oil/water and/or emulsion interfaces. As a result, the capability of surfactant residual measurement in both the oil and water phases enabled by the pyro-chemiluminescence method can provide additional insight on the level of affinity of the surfactant to the oil/water interface at different conditions.

TABLE 1

| | | | Calculated |
|---|---|---|---|
| | | | C12 quat |
| Sample | | N content | concentration |
| No. | Description | (ppm) | (ppm) |
| 17-34575 | toluene phase after | 0.2 | 3.5 |
| | partitioning of 100 ppm | | |
| | C12 quat 1% salinity | | |
| 17-34578 | o-xylene extract of brine | 3.1 | 77.1 |
| | phase after partition of 100 | | |
| | ppm C12 quat 1% salinity | | |
| 17-48174 | toluene phase after | 5.1 | 87.1 |
| | partition of 100 ppm C12 | | |
| | quat 24% salinity | | |
| 17-48176 | o-xylene extract of brine | 0.6 | 9.6 |
| | phase after partition of 100 | | |
| | ppm C12 quat 24% salinity | | |

### Example 2: N-(2-((2-hydroxyethyl)amino)ethyl)oleylamide (Abbreviated as Amide) Partitioning in Toluene and pH 5 Brine

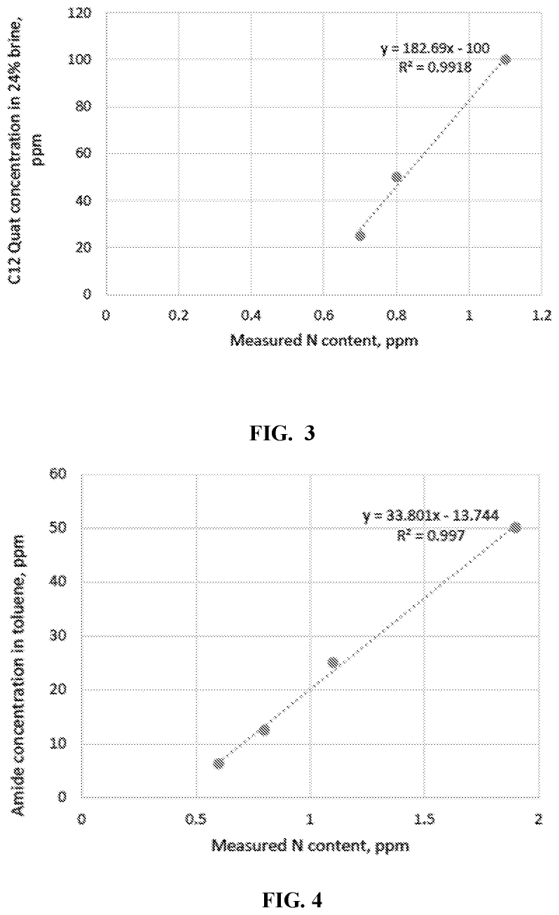

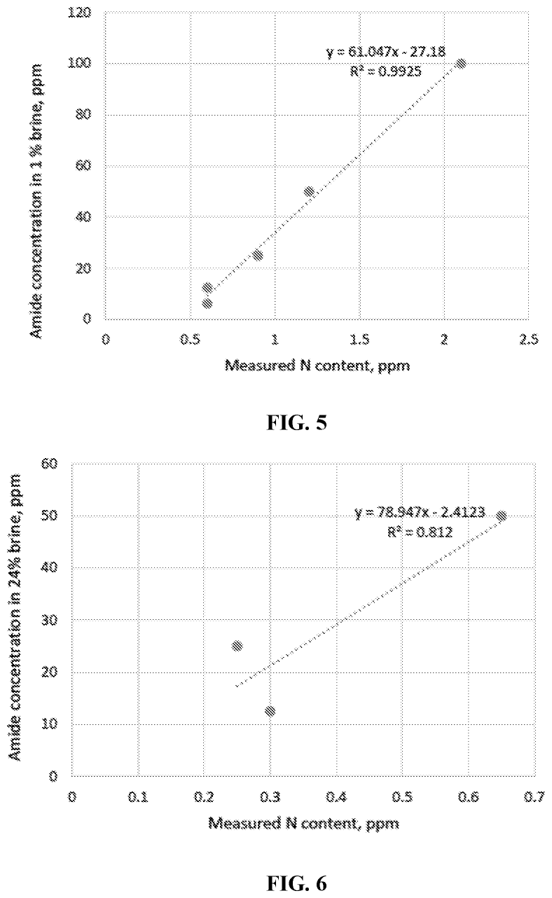

The partitioning experiments were conducted using the above procedure for 50 ppm amide in pH=5 brine with equal volume of toluene at ambient temperature. The calibration curves for amide in o-xylene, 1 wt. % NaCl and 24 wt. % NaCl are shown in FIGS. 4-6, respectively.

The measured nitrogen contents for the toluene and brine samples after partitioning are listed in Table 2. Corresponding amide concentrations are calculated based on this data and the corresponding calibrations in FIGS. 4-6. Unlike C12 quat, the partition of amide in the brine phase only moderately decreased with increasing salinity in the brine phase. This is reasonable and is attributed to the less ionic character of amide as compared to C12 quat.

TABLE 2

| | | | Calculated |
|---|---|---|---|
| | | | amide |
| Sample | | N content | concentration |
| No. | Description | (ppm) | (ppm) |
| 17-51201 | toluene phase after partition | 0.8 | 13.3 |
| | of 50 ppm amide in 1 wt. % | | |
| | brine | | |
| 17-51202 | o-xylene extract of brine | 1.0 | 35.2 |
| | phase after partition of 50 | | |
| | ppm amide in 1 wt. % brine | | |
| 17-58430 | toluene phase after partition | 0.7 | 9.9 |
| | of 50 ppm amide in 24 wt. % | | |
| | brine | | |
| 17-58431 | o-xylene extract of brine | 0.3 | 21.23 |
| | phase after partition of 50 | | |
| | ppm amide in 24 wt. % brine | | |

### Example 3: Commercial Corrosion Inhibitor Package EC1509A Partitioning in MPN Crude and MPN Brine

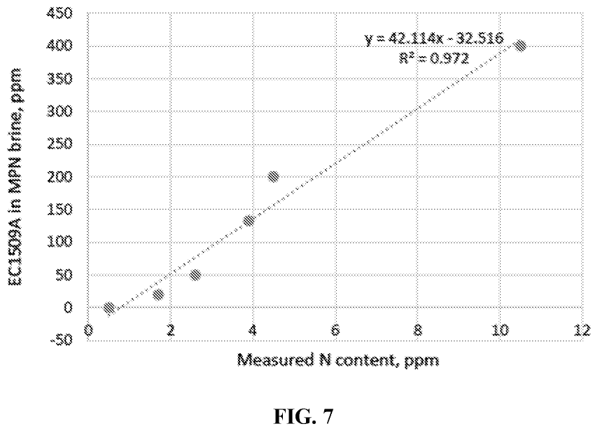

The partitioning experiments were conducted using the above procedure for 100 ppm Nalco EC1509A (concentration in total fluid (TF) by volume) in synthetic MPN (Mobil Producing Nigeria) brine with MPN crude at various ratios at ambient temperature. The active surfactants in EC1509A package are quaternary ammonium compounds. Because MPN crude itself contains a high content of nitrogen the majority of the measured nitrogen content would come from the oil phase itself after partitioning, therefore, only the brine phases were extracted and measured. The calibration curve for EC1509A in MPN brine is shown in FIG. 7. For references, partitioning experiments of blank MPN brine (no surfactant) with MPN crude were conducted under the same conditions. The measured nitrogen contents for the brine samples are listed in the Table 3. Corresponding EC1509A concentrations are calculated based on this data and the calibration in FIG. 7. The results demonstrate that surfactant residual concentration in the brine phase decreases with increasing water cut at a given surfactant injection concentration based on total fluid. Assuming all the surfactant goes into the brine phase, the calculated surfactant concentration in the brine will be 200 ppm at 50 vl. % water cut and 133.3 ppm at 75 vl. % water cut, respectively. Therefore, the observed trend of decreasing surfactant residual concentration in the brine with increasing water cut in this study is reasonable, given the fact that quaternary ammonium-based surfactants are water soluble chloride salts.

TABLE 3

| | | | Calculated |
|---|---|---|---|
| | | | surfactant |
| | | | residual |
| Sample | | N content | concentration |
| No. | Description | (ppm) | (ppm) |
| 17-44414 | o-xylene extract of no | 0.7 | negligible |
| | surfactant MPN brine | | |
| | partitioned with MPN crude, | | |
| | 50 vl. % water cut | | |
| 17-57103 | o-xylene extract of brine | 4.4 | 152.8 |
| | phase after partition of 100 | | |
| | ppm (TF) EC1509A in MPN | | |
| | brine with MPN crude, 50 vl. | | |
| | % water cut | | |
| 17-53531 | o-xylene extract of no | 0.7 | negligible |
| | surfactant MPN brine | | |
| | partitioned with MPN crude, | | |
| | 75 vl. % water cut | | |
| 17-54640 | o-xylene extract of brine | 1.2 | 18.0 |
| | phase after partition of 100 | | |
| | ppm (TF) EC1509A in MPN | | |
| | brine with MPN crude, 75 vl. | | |
| | % water cut | | |

All patents and patent applications, test procedures and other documents cited herein are fully incorporated by reference to the extent such disclosure is not inconsistent with this disclosure and for all jurisdictions in which such incorporation is permitted.

When numerical lower limits and numerical upper limits are listed herein, ranges from any lower limit to any upper limit are contemplated. While the illustrative embodiments of the disclosure have been described with particularity, it will be understood that various other modifications will be apparent to and can be readily made by those skilled in the art without departing from the spirit and scope of the disclosure. Accordingly, it is not intended that the scope of the claims appended hereto be limited to the examples and descriptions set forth herein but rather that the claims be construed as encompassing all the features of patentable novelty which reside in the present disclosure, including all features which would be treated as equivalents thereof by those skilled in the art to which the disclosure pertains.

The present disclosure has been described above with reference to numerous embodiments and specific examples. Many variations will suggest themselves to those skilled in the art in light of the above detailed description. All such obvious variations are within the full intended scope of the appended claims.

1. A method for determining the concentration of nitrogen and/or sulfur containing surfactants in oil and/or water phases of an oil and water system, comprising the steps of:
(a) partitioning the nitrogen and/or sulfur containing surfactant between the oil and water phase; and
(b) measuring the nitrogen and/or sulfur concentration in the oil and/or water phases by pyro-chemiluminescence and/or pyro-fluorescence.

2. The method according to claim 1, wherein the nitrogen concentration is measured using pyro-chemiluminescence.

3. The method according to claim 1, wherein the sulfur concentration is measured by pyro-luminesence.

4. The method according to claim 1, further comprising the step of separating at least a portion of the oil and/or water phases after partitioning the nitrogen and/or sulfur containing surfactant.

5. The method according to claim 1, further comprising the step of extracting at least a portion of the water phase with an organic solvent so as to transfer some or all of the nitrogen and/or sulfur containing surfactant to the organic solvent and measuring the nitrogen and/or sulfur concentration in the organic solvent.

6. The method according to claim 1, wherein the oil and water system is a produced oil and water system from an oil or gas well.

7. The method according to claim 1, wherein the water phase comprises one or more water soluble salts.

8. The method according to claim 7, wherein the water soluble salts are selected from alkali metal salts or alkaline earth salts

9. The method according to claim 8, wherein the alkali metal salts or alkaline earth salts are selected from halides, sulfates and bicarbonates.

10. The method according to claim 9, wherein the alkali metal salts or alkaline earth salts are selected from sodium chloride, potassium chloride, sodium bromide, calcium chloride, magnesium chloride, sodium sulfate, sodium bicarbonate and mixtures thereof.

11. The method according to claim 7, wherein the salt concentration is between about 0.1% and about 36% by weight.

12. The method according to claim 1, wherein the concentration of surfactant in the oil and water phases is, independently, between about 0.1 ppm and about 1000 ppm.

13. The method according to claim 1, wherein the volume ratio of oil to water in the oil and water system is between 0.1:1.0 and 1.0:0.1.

14. The method according to claim 5, wherein the volume ratio of organic solvent to water is between 0.1:1.0 and 1.0:0.1.

15. The method according to claim 5, wherein the organic solvent is selected from aliphatic and aromatic hydrocarbon solvents.

16. The method according to claim 1, wherein the surfactant is selected from the group consisting of non-ionic surfactant, ionic surfactant, amphoteric surfactant, and combinations thereof.

17. The method according to claim 16, wherein the non-ionic surfactant is selected from the group consisting of polyoxyethylene fatty acid amides, polyoxyethylene alkyl amines, alkylpyrrolidone, glucamides, mono- and dialkanol amides, polyoxyethylene alcohol mono- or diamides, alkylamine oxides and combinations thereof.

18. The method according to claim 16, wherein the ionic surfactant is selected from the group consisting of sulfonates, sulfates, ammonium and sulfonium alkylated quaternary or ternary compounds, singly or attached to polymeric compounds and combinations thereof.

19. The method according to claim 18 wherein the ionic surfactant is an anionic surfactant selected from the group consisting of sodium and ammonium long chain alkyl sulfonates and alkyl aryl sulfonates such as sodium dodecylbenzene sulfonate, dialkyl sodium sulfosuccinates, such as sodium dodecylbenzene sulfonate, dialkyl sodium sulfosuccinates, such as sodium bis-(2-ethylthioxy))-sulfosuccinate, alkyl sulfates such as sodium lauryl sulfate and combinations thereof.

20. The method according to claim 18, wherein the ionic surfactant is a cationic surfactant selected from the group consisting of quaternary ammonium compounds such as benzalkonium chloride, benzethonium chloride, cetrimonium bromide, stearyl dimethylbenzyl ammonium chloride, coconut amine and combinations thereof.

21. The method according to claim 19, wherein the anionic surfactant is selected from the group consisting of alkane sulfonate salts, alpha-olefin sulfonate salts, sulfonate salts of higher fatty acid esters, higher alcohol sulfate ester salts, fatty alcohol ether sulfates salts, condensates of higher fatty acids and amino acids, collagen hydrolysate derivatives and combinations thereof.

22. The method according to claim 20, wherein the cationic surfactant is selected from the group consisting of alkyltrimethylammonium salts, dialkyldimethylammonium salts, alkyldimethylbenzylammonium salts, alkylpyridinium salts, alkylisoquinolinium salts, benzethonium chloride, acylamino acid type cationic surfactants and combinations thereof.

23. The method according to claim 16, wherein the amphoteric surfactant is selected from the group consisting of amino acid, betaine, sultaine, imidazoline type amphoteric surfactants, soybean phospholipid, yolk lecithin and combinations thereof.

24. The method according to claim 16, wherein the amphoteric surfactant is selected from the group consisting of sodium N-dodecyl-beta-alanine, sodium N-lauryl-beta-iminodipropionate, myristoamphoacetate, lauryl betaine, laurylsulfobetaine and combinations thereof.
